(19) United States
(12) Patent Application Publication
(10) Pub. No.: US 2025/0135032 A1
(43) Pub. Date: May 1, 2025

(54) CRISPR METHODS FOR CORRECTING BAG3 GENE MUTATIONS IN VIVO

(71) Applicant: TEMPLE UNIVERSITY - OF THE COMMONWEALTH SYSTEM OF HIGHER EDUCATION, Philadelphia, PA (US)

(72) Inventors: Kamel KHALILI, Bala Cynwyd, PA (US); Chen CHEN, Philadelphia, PA (US); Shuren LIAO, Philadelphia, PA (US); Hong LIU, Philadelphia, PA (US)

(21) Appl. No.: 18/834,040

(22) PCT Filed: Jan. 30, 2023
(86) PCT No.: PCT/US2023/061594

§ 371 Date: Jul. 29, 2024

Related U.S. Application Data
(60) Provisional application No. 63/304,360, filed on Jan. 28, 2022.

(51) Int. Cl. A61K 48/00; A61K 38/46; C12N 15/11; C12N 15/86; C12Q 1/6883

(52) U.S. Cl. CPC A61K 48/005; A61K 38/465; C12N 15/11; C12N 15/86; C12Q 1/6883; C12N 2310/20; C12N 2750/14143; C12Q 2600/156

(57) ABSTRACT

Compositions include gene-editing complexes for the treatment of myopathies, cancer and neurodegenerative diseases. Specifically, the disclosure provides methods of identifying in a subject's biological sample, ‘at least one Bcl2-associated anthanogene 3 (BAGS) genetic mutation as compared to a control BAGS nucleic acid sequence, and administering to the subject a therapeutically effective amount of a gene-editing complex, wherein the gene-editing complex corrects the bag3 mutation to a wild-type bag3, thereby, treating the subject.

### CROSS REFERENCE TO RELATED APPLICATIONS

This application claims the benefit of priority under 35 U.S.C. § 119 (e) to U.S. Provisional Application No. 63/304,160 filed Jan. 28, 2022. The entire contents of this application are incorporated herein by reference in its entirety.

### BACKGROUND

A number of human diseases, including cardiac associated disorders and neurological disorders including Alzheimer's and other dementias may be associated with dysregulation of protein quality control. BAG3 is Hsp70 co-chaperone protein that plays a critical role in protein quality control. Dysregulation of BAG3 by mutations within the gene and/or low level expression of the protein may result in poor quality of several proteins that may cause protein aggregation and results in development of disease in brain and/or heart.

### SUMMARY

A genetic strategy based on CRISPR gene editing techniques is provided herein, for correcting mutations within the BAG3 gene to be applied in an in vivo system. Methods provided herein are noninvasive, precise, and scalable using a genetic approach. Advantages of this strategy is that it avoids risky procedures for overcoming disease, which in most cases the diseases cannot be cured.

Accordingly, in certain aspects, a method of diagnosing and treating a subject suspected of having a myopathy comprises identifying in a subject's biological sample, at least one Bcl2-associated athanogene 3 (BAG3) genetic mutation as compared to a control BAG3 nucleic acid sequence, wherein detection of certain genetic mutations is diagnostic of a myopathy, administering to the subject a therapeutically effective amount of a gene-editing complex, wherein the gene-editing complex corrects the bag3 mutation to a wild-type bag3, thereby, treating the subject. In certain embodiments, the mutation comprises insertions, deletions, truncation, substitutions or combinations thereof. In certain embodiments, the mutations encode for a mutated BAG3 polypeptide. In certain embodiments, the mutation comprises an E455K. In certain embodiments, the gene editing complex comprises comprising at least one isolated nucleic acid sequences wherein the isolated nucleic acid sequences encode a Clustered Regularly Interspaced Short Palindromic Repeat (CRISPR)-associated endonuclease and at least one guide RNA (gRNA), the gRNA being complementary to a target sequence in the bag3 gene. In certain embodiments, the at least one guide RNA comprises a sequence having at least a 90% sequence identity to SEQ ID NOS: 1, 2, or 3. In certain embodiments, the at least one guide RNA comprises sequences SEQ ID NOS: 1, 2, or 3.

In another aspect, a method of treating cancer comprises administering to the subject a therapeutically effective amount of an agent, wherein the agent modulates expression or amount of BAG3 molecules, proteins or peptides thereof in a target cell or tissue, as compared to a normal control, thereby treating cancer. In certain embodiments, increased amounts of BAG3 nucleic acids and/or BAG3 peptides as compared to normal controls are diagnostic of cancer. In certain embodiments, the cancer comprises melanomas, glioblastomas or adenocarcinomas. In certain embodiments, the agent comprises miRNA, dsDNA, IncRNA, siRNA, short hairpin RNAs (shRNAs), antisense oligonucleotide, a phosphorodiamidate morpholino oligomer (PMO), a peptide-conjugated phosphorodiamidate morpholino oligomer (PPMO), ribozymes, gene-editing complexes, or combinations thereof. In certain embodiments, the gene editing complex comprises comprising at least one isolated nucleic acid sequences wherein the isolated nucleic acid sequences encode a Clustered Regularly Interspaced Short Palindromic Repeat (CRISPR)-associated endonuclease and at least one guide RNA (gRNA), the gRNA being complementary to a target sequence in the bag3 gene. In certain embodiments, the gene-editing agent is targeted to bag3 gene, bag3 gene regulatory elements or the combination thereof. In certain embodiments, the gene regulatory elements comprise promoters, enhancers, initiation codons, stop codons, polyadenylation signals or combinations thereof.

In another aspect, a method of treating preventing or treating neurodegeneration in a subject, comprises administering Bcl2-associated athanogene 3 (BAG3) polynucleotide, polypeptide and/or agents which induce BAG3 expression or function. In certain embodiments, the agent comprises miRNA, dsDNA, IncRNA, siRNA, short hairpin RNAs (shRNAs), antisense oligonucleotide, a phosphorodiamidate morpholino oligomer (PMO), a peptide-conjugated phosphorodiamidate morpholino oligomer (PPMO), ribozymes, gene-editing complexes, proteins or peptides thereof, peptidomimetics, small molecules, organic or inorganic compounds, synthetic or natural compounds, or combinations thereof. In certain embodiments, the gene editing complex comprises comprising at least one isolated nucleic acid sequences wherein the isolated nucleic acid sequences encode a Clustered Regularly Interspaced Short Palindromic Repeat (CRISPR)-associated endonuclease and at least one guide RNA (gRNA), the gRNA being complementary to a target sequence in the bag3 gene. In certain embodiments, the gene-editing agent is targeted to bag3 gene, bag3 gene regulatory elements or the combination thereof. In certain embodiments, the gene regulatory elements comprise promoters, enhancers, initiation codons, stop codons, polyadenylation signals or combinations thereof. In certain embodiments, the agent comprises an expression vector expressing a BAG3 protein or active fragments thereof, oligonucleotides or combinations thereof. In certain embodiments, the expression vector comprises a viral vector, tropic vector, plasmid, or a yeast vector. In certain embodiments, a neurotropic vector, a cardiotropic vector comprises an adenovirus vector, an adeno-associated virus vector (AAV), a coxsackie virus vector, cytomegalovirus vector, Epstein-Barr virus vector, parvovirus vector, or hepatitis virus vectors.

In certain embodiments, a BAG3 mutation comprises an E455K mutation.

In certain embodiments, an SaCas9 guide comprises a mutation at amino acid 455 comprising GTGATCGAAGAGTATTTGACCAAAGAGC (SEQ ID NO: 1; 455E; underlined bases AAGAG (SEQ ID NO: 4) represents the PAM sequence), GTGATCAAAGAGTATTTGACCAAAGAGC (SEQ ID NO: 2; 455K; underlined bases AAGAG (SEQ ID NO: 4) represents the PAM sequence), TGATCAAAGAGTATTTGACCA (BAG3 455K; SEQ ID NO: 3), or the combination thereof.

In certain embodiments, a PAM sequence comprises AAGAG (SEQ ID NO: 4).

In certain embodiments, neurological disorders comprise: AIDS dementia complex, Alzheimer's disease, amyotrophic lateral sclerosis, adrenoleukodystrophy, Alexander disease, Alper's disease, ataxia telangiectasia, Batten disease, bovine spongiform encephalopathy (BSE), Canavan discase, corticobasal degeneration, Creutzfeldt-Jakob disease, dementia with Lewy bodies, fatal familial insomnia, frontotemporal lobar degeneration, Huntington's disease, Kennedy's discase, Krabbe disease, Lyme disease, Machado-Joseph disease, multiple sclerosis, multiple system atrophy, neuroacanthocytosis, Niemann-Pick disease, Parkinson's disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, or wobbly hedgehog syndrome.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “AAV” refers to adeno-associated virus and may be used to refer to the naturally occurring wild-type virus itself or derivatives thereof. The term covers all subtypes, serotypes and pseudotypes, and both naturally occurring and recombinant forms, except where required otherwise. The AAV genome is built of single stranded DNA and comprises inverted terminal repeats (ITRs) at both ends of the DNA strand, and two open reading frames: rep and cap, encoding replication and capsid proteins, respectively. A foreign polynucleotide can replace the native rep and cap genes. AAVs can be made with a variety of different serotype capsids which have varying transduction profiles or as used herein, “tropism” for different tissue types. As used herein, the term “serotype” refers to an AAV which is identified by and distinguished from other AAVs based on capsid protein reactivity with defined antisera, e.g., AAV serotypes 1, 2, 3, 4, 5, 6, 7, 8, 9, 10, 11, 12, 13, 14, 15, 16, DJ or DJ/8. For example, serotype AAV2 is used to refer to an AAV which contains capsid proteins encoded from the cap gene of AAV2 and a genome containing 5′ and 3′ ITR sequences from the same AAV2 serotype. Pseudotyped AAV as refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped rAAV are produced using standard techniques described in the art. In certain embodiments, the AAVs comprise mutations in the capsid proteins. These mutations comprise one or more amino acid changes at one or multiple locations of the capsid proteins.

As used herein, “Bcl-2 associated athanogene” (BAG) are inclusive of all family members e.g. BAG1, BAG2, BAG3, etc., isoforms, mutants, cDNA sequences, alleles, fragments, species, coding and noncoding sequences, sense and antisense polynucleotide strands, etc.

As used herein “BAG3”, “BAG3 molecules”, “BCL2-associated athanogene 3 (BAG3) genes”, “BCL2-associated athanogene 3 (BAG3) molecules” are inclusive of all family members, isoforms, mutants, cDNA sequences, alleles, fragments, species, coding and noncoding sequences, sense and antisense polynucleotide strands, etc. (HGNC (939) Entrez Gene (9531) Ensembl (ENSG00000151929) OMIM (603883) UniProtKB (095817)). Similarly, “BAG3”, “BAG3 molecules”, “BCL2-associated athanogene 3 (BAG3) molecules” also refer to BAG3 polypeptides or fragment thereof, proteins, variants, derivatives etc. The term “molecule” thus encompasses both the nucleic acid sequences and amino acid sequences of BAG3.

As used herein, “biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils, thymus and skin, or samples taken from tumors. Examples of “body fluid samples” include, but are not limited to blood, serum, semen, prostate fluid, seminal fluid, urine, feces, saliva, sputum, mucus, bone marrow, lymph, and tears.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements-or, as appropriate, equivalents thereof-and that other elements can be included and still fall within the scope/definition of the defined item, composition, apparatus, method, process, system, etc.

As used herein, the term “crRNA” or “guide RNA” or “single guide RNA” or “sgRNA” or “one or more nucleic acid components” of a CRISPR-Cas comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide RNA (gRNA) is a chimeric molecule that consists of tracrRNA and crRNA, anteceded by an 18-20-nt spacer sequence complementary to target DNA before a protospacer adjacent motif (PAM). In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide RNA may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (IRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (IncRNA), and small cytoplasmic RNA (scRNA). In some embodiments, the target sequence may be a sequence within a RNA molecule selected from the group consisting of mRNA, pre-mRNA, and rRNA. In some embodiments, the target sequence may be a sequence within an RNA molecule selected from the group consisting of ncRNA, and IncRNA. In some embodiments, the target sequence may be a sequence within an mRNA molecule or a pre-mRNA molecule.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residue in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23. 24, 25, 30, 35, 40, 45, 50, or more nucleotides, or refers to two nucleic acids that hybridize under stringent conditions.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogsteen binding, or in any other sequence specific manner. The complex may comprise two strands forming a duplex structure, three or more strands forming a multi stranded complex, a single self-hybridizing strand, or any combination of these.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

“Protospacer adjacent motif” (PAM) is a 3-nt sequence located immediately downstream of the single guide RNA (sgRNA) target site, which plays an essential role in binding and for Cas-mediated DNA cleavage. The PAMs are the various extended conserved bases at the 5′ or 3′ end of the protospacer.

The term “stringent conditions” for hybridization refers to conditions under which a nucleic acid having complementarily to a target sequence predominantly hybridizes with the target sequence, and substantially does not hybridize to non-target sequences. Stringent conditions are generally sequence-dependent and vary depending on a number of factors, in general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology—Hybridization With Nucleic Acid Probes Part 1, Second Chapter “Overview of principles of hybridization and the strategy of nucleic acid probe assay”, Elsevier, N.Y.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s)

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrastemal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “percent sequence identity” or having “a sequence identity” refers to the degree of identity between any given query sequence and a subject sequence.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “therapeutic” treatment is a treatment administered to a subject who exhibits signs of pathology, for the purpose of diminishing or eliminating those signs.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

“Variant” as the term is used herein, is a nucleic acid sequence or a peptide sequence that differs in sequence from a reference nucleic acid sequence or peptide sequence respectively but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Where any a nucleic acid sequence or an amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human. Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is a schematic representation of a construct of generation I BAG3 base editor.

FIG. 2 is a schematic representation of a construct of generation I BAG3 base editor.

FIG. 3 shows MEF cells of BAG3 E455K heterozygote mutant.

FIG. 4 shows results from MEF cells transfected with generation I BAG3 base editor.

FIG. 5 shows results from MEF cells transfected with generation II BAG3 base editor.

### DETAILED DESCRIPTION

The following description of certain embodiments is merely exemplary in nature and is in no way intended to limit the invention, its application or uses. Embodiments of the invention may be practiced without the theoretical aspects presented. Moreover, the theoretical aspects are presented with the understanding that Applicant does not seek to be bound by the theory presented.

The human bag3 gene is located on the long arm of chromosome 10 at position 26.11 (10q26.11). Mutations in the bag3 gene are associated with the development of severe diseases, such as for example, myopathies, cancer Age-Related Neurodegenerative Disorders. Examples of myopathies include myofibrillar myopathy or dilated cardiomyopathy. The HSP70 co-chaperone BAG3 is found to be upregulated in many human cancers of various origins, for instance in melanomas, glioblastomas, or pancreatic adenocarcinomas. Age-Related Neurodegenerative Disorders such as Amyotrophic Lateral Sclerosis (also known as ALS, Lou Gehrig's disease, or motor neuron disease), Huntington's disease (HD), Alzheimer's disease (AD). The BAG3-pathway is also able to remove disease-associated aggregation-prone proteins, such as mutated SOD1 or polyQ43-huntingtin, and is thereby linked to neuroprotection in age-related neurodegenerative disorders, like ALS, HD, or AD.

BAG proteins are characterized by a common conserved region located near the C terminus, termed the BAG domain (BD) that mediates direct interaction with the ATPase domain of Hsp70/Hsc70 molecular chaperones. Six BAG family members have been identified in humans and shown to regulate, both positively and negatively, the function of Hsp 70/Hsc70, and to form complexes with a range of transcription factors modulating various physiological processes including apoptosis, tumorigenesis, neuronal differentiation, stress responses, and the cell cycle. In addition to the conserved BD, several other domains within the BAG proteins have been identified and are likely to modulate both target specificity and BAG protein localization within cells. The BAG proteins generally differ in the N-terminal region, which imparts specificity to particular proteins and pathways. Ubiquitin-like domain at the N terminus of human BAG proteins (BAG1 and BAG6) is probably functionally relevant and conserved in yeast, plants, and worms. BAG proteins regulate diverse physiological processes in animals, including apoptosis, tumorigenesis, neuronal differentiation, stress responses, and the cell cycle.

The six human BAG proteins identified are BAG-1 (RAP46/HAP46), BAG-2,BAG-3 (CAIR-1/Bis), BAG-4 (SODD), BAG-5, and BAG-6 (BAT3/Scythe), and all share the signature BD near the C-terminal end, with the exception of BAG 5, which contains four of such domains.

The NCBI reference amino acid sequence for BAG3 can be found at Genbank under accession number NP_004272.2; Public GI: 14043024. The NCBI reference nucleic acid sequence for BAG3 can be found at Genbank under accession number NM 004281.3 GI: 62530382. Other BAG3 amino acid sequences include, for example, without limitation, 095817.3 GI: 12643665; EAW49383.1 GI: 119569768; EAW49382.1 GI: 119569767; and CAE55998.1 GI: 38502170. The BAG3 polypeptide of the invention can be a variant of a polypeptide described herein, provided it retains functionality.

In certain embodiments, a therapeutic agent for treatment of subjects modulates the expression or amounts of BAG, e.g. BAG3 in a cell. In certain embodiments, a therapeutic agent for treatment of subjects increases the expression or amounts of BAG, e.g. BAG3 in a cell. In some embodiments, compositions comprise nucleic acid sequences complementary to BCL2-associated athanogene 3 (BAG3), including without limitation, cDNA, sense and/or antisense sequences of BAG3.

In another embodiment, it may be necessary to increase the expression of BAG3 comprising one or more variants in a cell or patient by oligonucleotides that modulate the expression of BAG3, for example, transcriptional regulator elements. In a preferred embodiment, an oligonucleotide comprises at least five consecutive bases complementary to a nucleic acid sequence, wherein the oligonucleotide specifically hybridizes to a target sequence and modulates expression of BAG3 in vivo or in vitro. In another embodiment, the oligonucleotides include variants in which a different base is present at one or more of the nucleotide positions in the compound. For example, if the first nucleotide is an adenosine, variants may be produced which contain thymidine, guanosine or cytidine at this position. This may be done at any of the positions of the oligonucleotide. These compounds are then tested using the methods described herein to determine their ability to inhibit expression of a target nucleic acid.

Accordingly, in certain embodiments, a method of diagnosing and treating a patient having cardiac disease, comprises identifying in a patient sample, at least one Bcl2-associated athanogene 3 (BAG3) genetic variant as compared to a control BAG3 nucleic acid sequence, wherein detection of certain variants are predictive of whether an increase in BAG3 levels is therapeutic for the patient, and, administering to the patient identified as having such a variant, a therapeutically effective amount of an agent wherein the agent modulates expression or amount of BAG3 molecules, proteins or peptides thereof in a target cell or tissue, as compared to a normal control.

In certain embodiments, a composition comprises a gene-editing complex, wherein mutations to bag3 are edited to correct the mutations to wild type BAG3. Examples of bag3 mutations include E455K. In certain embodiments, the gene editing complex comprises an SaCas9 guide comprises a mutation at amino acid 455 comprising GTGATCGAAGAGTATTTGACCAAAGAGC (SEQ ID NO: 1; 455E; underlined bases AAGAG (SEQ ID NO: 4) represents the PAM sequence), GTGATCAAAGAGTATTTGACCAAAGAGC (SEQ ID NO: 2; 455K; underlined bases AAGAG (SEQ ID NO: 4) represents the PAM sequence), TGATCAAAGAGTATTTGACCA (BAG3 455K; SEQ ID NO: 3), or the combination thereof.

### Gene Editing Complexes

The RNA-guided Cas9 biotechnology induces genome editing without detectable off-target effects. This technique takes advantage of the genome defense mechanisms in bacteria that CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR. (Clustered Regularly Interspaced Short Palindromic Repeats) RNA (crRNA). Cas9 belongs to the type II CRISPR/Cas system and has strong endonuclease activity to cut target DNA.

Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA: tracrRNA duplex directs Cas9 to target DNA via complementary base pairing between the spacer on the crRNA and the complementary sequence (called the protospacer) on the target DNA (tDNA). Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (gRNA) via a synthetic stem loop (AGAAAU) to mimic the natural crRNA/tracrRNA duplex. Such gRNA, like shRNA, can be synthesized or in vitro transcribed for direct RNA transfection or expressed from a RNA expression vector (e.g., U6 or HI promoter-driven vectors). Therefore, the Cas9 gRNA technology requires the expression of the Cas9 protein and gRNA, which then form a gene editing complex at the specific target DNA binding site within the target genome and inflict cleavage/mutation of the target DNA.

Cas9 variant system: An NGG PAM at the 3′ end of the target DNA site is essential for the recognization and cleavage of the target gene by Cas9 protein. Besides classical NGG PAM sites, other PAM sites such as NGA and NAG also exist, but their efficiency of genome editing is not high. However, such PAM sites only exist in about one-sixteenth of the human genome, thereby largely restricting the targetable genomic loci. For this purpose, several Cas9 variants have been developed to expand PAM compatibility.

In 2018, David Liu et al. (Hu J. H., Miller S. M., Geurts M. H., Tang W., Chen L. Evolved Cas9 variants with broad PAM compatibility and high DNA specificity. Nature. 2018;556:57-63) developed xCas9 by phage-assisted continuous evolution (PACE), which can recognize multiple PAMs (NG, GAA, GAT, etc.). In the latter half of the same year, Nishimasu et al. developed SpCas9-NG, which can recognize relaxed NG PAMs (Nishimasu H., Shi X., Ishiguro S., Gao L., Hirano S. Engineered CRISPR-Cas9 nuclease with expanded targeting space. Science. 2018;361:1259-1262). In 2020, Miller et al. developed three new SpCas9 variants recognizing non-G PAMs, such as NRRH, NRCH and NRTH PAMs (Miller S. M., Wang T., Randolph P. B., Arbab M., Shen M. W. Continuous evolution of SpCas9 variants compatible with non-G PAMs. Nat Biotechnol. 2020;38:471-481). Later in the same year, Walton et al. developed a SpCas9 variant named SpG, which is capable of targeting an expanded set of NGN PAMs (Walton R. T., Christie K. A., Whittaker M. N., Kleinstiver B. P. Unconstrained genome targeting with near-PAMless engineered CRISPR-Cas9 variants. Science. 2020,368:290-296). Subsequently, they optimized the SpG system and developed a near-PAMless variant named SpRY, which is capable of editing nearly all PAMs (NRN and NYN PAMs).

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cpfl to edit the target site of interest.

The CRISPR-Cas systems of bacterial and archaeal adaptive immunity show extreme diversity of protein composition and genomic loci architecture. The CRISPR-Cas system loci have more than 50 gene families and there are no strictly universal genes indicating fast evolution and extreme diversity of loci architecture. So far, adopting a multi-pronged approach, there is comprehensive Cas gene identification of about 395 profiles for 93 Cas proteins. Classification includes signature gene profiles plus signatures of locus architecture. A new classification of CRISPR-Cas systems is proposed in which these systems are broadly divided into two classes, Class 1 with multi-subunit effector complexes and Class 2 with single-subunit effector modules exemplified by the Cas9 protein.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains. Active DNA-targeting CRISPR-Cas systems use 2 to 4 nucleotide protospacer-adjacent motifs (PAMs) located next to target sequences for self-versus non-self-discrimination. ARMAN-1 has a strong ‘NGG’ PAM preference. Cas9 also employs two separate transcripts, CRISPR RNA (crRNA) and trans-activating CRISPR RNA (tracrRNA), for RNA-guided DNA cleavage. Putative tracrRNA was identified in the vicinity of both ARMAN-1 and ARMAN-4 CRISPR-Cas9 systems (Burstein, D. et al. New CRISPR-Cas systems from uncultivated microbes. Nature. 2017 Feb. 9;542 (7640): 237-241. doi: 10.1038/nature21059. Epub 2016 Dec. 22).

In embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein. As used herein, the term “Cas” is meant to include all Cas molecules comprising variants, mutants, orthologues, homologues, high-fidelity variants and the like.

As described herein, CRISPR-Cas systems generally refer to an enzyme system that includes a guide RNA sequence that contains a nucleotide sequence complementary or substantially complementary to a region of a target polynucleotide, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, the CRISPR/Cas system used herein can be a type I, a type II, or a type III system. Non-limiting examples of suitable CRISPR/Cas proteins include Cas3, Cas4, Cas5, Casse (or CasD), Cas6, Cas6e, Cas6f, Cas7, Cas8a1, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csz1, Csx15, Csf1, Csf2, Csf3, Csf4, and Cui 966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), CasB (e.g., Cas13a, Cas13b (such as Cas13b-t1, Cas13b-t2,Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas 12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ.

Recently, the Class 2 type VI single-component CRISPR-Cas effector Cas 13a, previously known as C2c2 (Shmakov et al. (2015) “Discovery and Functional Characterization of Diverse Class 2 CRISPR-Cas Systems”; Molecular Cell 60:1-13; doi: dx.doi.org/10.1016/j.molcel.2015.10.008) was characterized as an RNA-guided RNAse (Abudayyeh et al. (2016), Science, [Epub ahead of print], June 2; “C2c2 is a single-component programmable RNA-guided RNA-targeting CRISPR effector”; doi: 10.1126/science.aaf5573). It was demonstrated that C2c2 (e.g. from Leptotrichia shahii) provides robust interference against RNA phage infection. Through in vitro biochemical analysis and in vivo assays, it was shown that C2c2 can be programmed to cleave ssRNA targets carrying protospacers flanked by a 3′H (non-G) PAM. Cleavage is mediated by catalytic residues in the two conserved HEPN domains of C2c2, mutations in which generate a catalytically inactive RNA-binding protein. C2c2 is guided by a single crRNA and can be re-programmed to deplete specific mRNAs in vivo. It was shown that LshC2c2 can be targeted to a specific site of interest and can carry out non-specific RNase activity once primed with the cognate target RNA.

C2c2 is now known as Cas 13a. It will be understood that the term “C2c2” herein is used interchangeably with “Cas13a”. As used herein, Cas13 may refer to Cas13a or Cas 13b or Cas 13c or Cas 13d or other member in the Cas 13 family. In certain embodiments, the compositions comprise Cas 13. In certain embodiments, the Cas 13 comprises Cas 13a. In certain embodiments, the Cas 13 comprises Cas 13b. In certain embodiments, the Cas 13 comprises Cas 13c. In certain embodiments, the Cas 13 comprises Cas13d.

Cas 13a is the first naturally occurring CRISPR system that targets only RNA. The Class 2 type VI-A CRISPR-Cas effector “Cast 3a” demonstrates an RNA-guided RNase function. Cas13a from the bacterium Leptotrichia shahii provides interference against RNA phage. In vitro biochemical analysis shows that Cas 13a is guided by a single crRNA and can be programmed to cleave ssRNA targets carrying complementary protospacers. In bacteria, Cas13a can be programmed to knock down specific mRNAs. Cleavage is mediated by catalytic residues in the two conserved HEPN domains, mutations in which generate catalytically inactive RNA-binding proteins. These results demonstrate the capability of Cas13a as a new RNA-targeting tools.

Cas13a can be programmed to cleave particular RNA sequences in bacterial cells. The RNA-focused action of Cas13a complements the CRISPR-Cas9 system, which targets DNA, the genomic blueprint for cellular identity and function. The ability to target only RNA, which helps carry out the genomic instructions, offers the ability to specifically manipulate RNA in a high-throughput manner-and manipulate gene function more broadly.

In certain embodiments, the Cas13 comprises a catalytically inactive Cas effector protein (e.g., dCas13). The catalytically inactive Cas 13 (dCas13) may include truncations of Cas 13 proteins, e.g., at the C-terminus, the N-terminus, or both. The dCas13 may be a catalytically inactive form of any Cas 13 subtype protein. For example, dCas13 may be dCas13a, dCas13b, dCas13c, or dCas13d. In certain embodiments, the dCas13 may be modified Cas13 effector proteins from Prevotella sp. P5-125, Riemerella anatipestifer, or Porphyromonas gulae.

In certain embodiments, the composition comprises orthologues of Cas13. The terms “orthologue” and “homologue” are well known in the art. By means of further guidance, a “homologue” of a protein as used herein is a protein of the same species which performs the same or a similar function as the protein it is a homologue of Homologous proteins may but need not be structurally related or are only partially structurally related. An “orthologue” of a protein as used herein is a protein of a different species which performs the same or a similar function as the protein it is an orthologue of. Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22 (4): 359-66. doi: 10.1002/pro.2225.). See also Shmakov et al. (2015) for application in the field of CRISPR-Cas loci. Homologous proteins may but need not be structurally related or are only partially structurally related.

The Cas 13 gene is found in several diverse bacterial genomes, typically in the same locus with cas1, cas2, and cas4 genes and a CRISPR cassette (for example, FNFX1_1431-FNFX1_1428 of Francisella cf. novicida Fxl). Thus, the layout of this putative novel CRISPR-Cas system appears to be similar to that of type II-B. Furthermore, similar to Cas9, the Cas13protein contains a readily identifiable C-terminal region that is homologous to the transposon ORF-B and includes an active RuvC-like nuclease, an arginine-rich region, and a Zn finger (absent in Cas9). However, unlike Cas9, Cas13 is also present in several genomes without a CRISPR-Cas context and its relatively high similarity with ORF-B suggests that it might be a transposon component. It was suggested that if this was a genuine CRISPR-Cas system and Cas13 is a functional analog of Cas9 it would be a novel CRISPR-Cas type, namely type V (See Annotation and Classification of CRISPR-Cas Systems. Makarova K S, Koonin E V. Methods Mol Biol. 2015; 1311:47-75).

Cas13 has several advantages over other Cas proteins, e.g. Cas9. RNA editing doesn't require homology-directed repair (HDR) machinery and could thus Cas 13 can be used in non-dividing cells. Cas 13 enzymes also do not require a PAM sequence at the target locus, making them more flexible than Cas9/Cpfl. Some Cas13 enzymes prefer targets with a given single base protospacer flanking site (PFS) sequence, but orthologues like LwaCas13a do not require a specific PFS.

In certain embodiments, the Cas 13 protein is from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Leptospira, Desulfovibrio, Desulfonatronum, Opitutaceae, Tuberibacillus, Bacillus, Brevibacilus, Methylobacterium, Butyvibrio, Perigrinibacterium, Pareubacterium, Moraxella, Thiomicrospira or Acidaminococcus.

In certain embodiments, the Cas 13 protein is from an organism comprising S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii, L inadai, F. tularensis 1, P. albensis, L. bacterium, B. proteoclasticus, P. bacterium, P. crevioricanis, P. disiens and P. macacae.

In certain embodiments, the Cas 13 may comprise a chimeric protein comprising a first fragment from a first protein (e.g., a Cas 13) orthologue and a second fragment from a second (e.g., a Cas13) protein orthologue, and wherein the first and second protein orthologues are different. At least one of the first and second protein (e.g., a Cas13) orthologues may comprise a Cast 3 from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Butyvibrio, Perigrinibacterium, Pareubacterium, Moraxella, Thiomicrospira or Acidaminococcus. In certain embodiments, a chimeric protein comprising a first fragment and a second fragment wherein each of the first and second fragments is selected from a Cas13 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Butyvibrio, Perigrinibacterium, Pareubacterium, Moraxella, Thiomicrospira or Acidaminococcus wherein the first and second fragments are not from the same bacteria; for instance a chimeric effector protein comprising a first fragment and a second fragment wherein each of the first and second fragments is selected from a Cas13 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis l, Prevotella albensis, Lachnospiraceae bacterium MC20171, Butyrivibrio proteoclasticus, Peregrinibacteria bacterium GW201 1_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria. In certain embodiments, the Cast 3 protein may be an orthologue of an organism of a genus which includes, but is not limited to Acidaminococcus sp, Lachnospiraceae bacterium or Moraxella bovoculi. In certain embodiments, the type V Cas protein may be an orthologue of an organism of a species which includes but is not limited to Acidaminococcus sp. BV3L6; Lachnospiraceae bacterium ND2006 (LbCas13) or Moraxella bovoculi 237. In certain embodiments, the homologue or orthologue of Cas 13 as referred to herein has a sequence homology or identity of at least 80%, or at least 85%, or at least 90%, such as for instance at least 95% with the wild type FnCas13, AsCas13 or LbCas13.

In certain embodiments, the Cas peptide is Cas9. The CRISPR-associated endonuclease Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Pseudomonas aeruginosa, Escherichia coli, or other sequenced bacteria genomes and archaea, or other prokaryotic microorganisms may also be a source of the Cas9 sequence utilized in the embodiments disclosed herein.

The wild type Streptococcus pyogenes Cas9 sequence can be modified. The nucleic acid sequence can be codon optimized for efficient expression in plant cells Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GI: 669193757; KM099232.1 GI: 669193761; or KM099233.1 GI: 669193765 or Cas9 amino acid sequence of PX330 or PX260 (Addgene, Cambridge, MA). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution e.g., a conservative amino acid substitution). For example, a biologically active variant of a Cas9 polypeptide can have an amino acid sequence with at least or about 50% sequence identity e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as nonstandard amino acids (e.g., amino acids having the D-configuration instead of the L-configuration). The present peptides can also include amino acid residues that are modified versions of standard residues (e.g., pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine (2R,3S)-2-amino-3-methylpentanoic acid and L-cyclopentyl glycine(S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

The Cas9 nuclease sequence can be a mutated sequence. For example, the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the nontarget strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOE10.1126/science.aad5227).

In some embodiments, SpCas9 variants comprise one or more point mutations, including, but not limited to R780A, K810A, K848A, K855A, H982A, K1003A, and R1060A (Slaymaker et al., 2016, Science, 351 (6268): 84-88). In some embodiments, SpCas9 variants comprise DI 135E point mutation (Kleinstiver et al., 2015, Nature, 523 (7561): 481-485). In some embodiments, SpCas9 variants comprise one or more point mutations, including, but not limited to N497A, R661A, Q695A, Q926A, DI 135E, L169A, and Y450A (Kleinstiver et al., 2016, Nature, doi: 10.1038/naturel6526). In some embodiments, SpCas9 variants comprise one or more point mutations, including but not limited to M495A, M694A, and M698A. Y450 is involved with hydrophobic base pair stacking. N497, R661, Q695, Q926 are involved with residue to base hydrogen bonding contributing to off-target effects. N497 hydrogen bonding through peptide backbone. L169A is involved with hydrophobic base pair stacking. M495A, M694A, and H698A are involved with hydrophobic base pair stacking.

In some embodiments, SpCas9 variants comprise one or more point mutations at one or more of the following residues: R780, K810, K848, K855, H982, KI 003, R1060, DI 135, N497, R661, Q695, Q926, L169, Y450, M495, M694, and M698. In some embodiments, SpCas9 variants comprise one or more point mutations selected from the group of: R780A, K810A, K848A, K855A, H982A, K1003A, R1060A, DI 135E, N497A, R661A, Q695A, Q926A, L169A, Y450A, M495A, M694A, and M698A.

In some embodiments, the SpCas9 variant comprises the point mutations, relative to wildtype SpCas9, of N497A, R661A, Q695A, and Q926A. In some embodiments, the SpCas9 variant comprises the point mutations, relative to wildtype SpCas9, of N497A, R661A, Q695A, Q926A, and DI 135E. In some embodiments, the SpCas9 variant comprises the point mutations, relative to wildtype SpCas9, of N497A, R661A, Q695A, Q926A, and L169A. In some embodiments, the SpCas9 variant comprises the point mutations, relative to wildtype SpCas9, of N497A, R661A, Q695A, Q926A, and Y450A. In some embodiments, the SpCas9 variant comprises the point mutations, relative to wildtype SpCas9, of N497A, R661A, Q695A, Q926A, and M495A. In some embodiments, the SpCas9 variant comprises the point mutations, relative to wildtype SpCas9, of N497A, R661A, Q695A, Q926A, and M694A. In some embodiments, the SpCas9 variant comprises the point mutations, relative to wildtype SpCas9, of N497A, R661A, Q695A, Q926A, and H698A. In some embodiments, the SpCas9 variant comprises the point mutations, relative to wildtype SpCas9, of N497A, R661A, Q695A, Q926A, DI 135E, and L169A. In some embodiments, the SpCas9 variant comprises the point mutations, relative to wildtype SpCas9, of N497A, R661A, Q695A, Q926A, DI 135E, and Y450A. In some embodiments, the SpCas9 variant comprises the point mutations, relative to wildtype SpCas9, of N497A, R661A, Q695A, Q926A, DI 135E, and M495A. In some embodiments, the SpCas9 variant comprises the point mutations, relative to wildtype SpCas9, of N497A, R661A, Q695A, Q926A, D1135E, and M694A. In some embodiments, the SpCas9 variant comprises the point mutations, relative to wildtype SpCas9, of N497A, R661A, Q695A, Q926A, DI 135E, and M698A.

In some embodiments, the SpCas9 variant comprises the point mutations, relative to wildtype SpCas9, of R661A, Q695A, and Q926A. In some embodiments, the SpCas9 variant comprises the point mutations, relative to wildtype SpCas9, of R661A, Q695A, Q926A, and DI 135E. In some embodiments, the SpCas9 variant comprises the point mutations, relative to wildtype SpCas9, of R661A, Q695A, Q926A, and L169A. In some embodiments, the SpCas9 variant comprises the point mutations, relative to wildtype SpCas9, of R661A, Q695A, Q926A, and Y450A. In some embodiments, the SpCas9 variant comprises the point mutations, relative to wildtype SpCas9, of R661A, Q695A, Q926A, and M495A. In some embodiments, the SpCas9 variant comprises the point mutations, relative to wildtype SpCas9, of R661A, Q695A, Q926A, and M694A. In some embodiments, the SpCas9 variant comprises the point mutations, relative to wildtype SpCas9, of R661A, Q695A, Q926A, and H698A. In some embodiments, the SpCas9 variant comprises the point mutations, relative to wildtype SpCas9, of R661A, Q695A, Q926A, DI 135E, and L169A. In some embodiments, the SpCas9 variant comprises the point mutations, relative to wildtype SpCas9, of R661A, Q695A, Q926A, DI 135E, and Y450A. In some embodiments, the SpCas9 variant comprises the point mutations, relative to wildtype SpCas9, of R661A, Q695A, Q926A, DI 135E, and M495A. In some embodiments, the SpCas9 variant comprises the point mutations, relative to wildtype SpCas9, of R661A, Q695A, Q926A, DI 135E, and M694A. In some embodiments, the SpCas9 variant comprises the point mutations, relative to wildtype SpCas9, of R661A, Q695A, Q926A, DI 135E, and M698A.

Three variants found to have the best cleavage efficiency and fewest off-target effects: SpCas9 (K855A), SpCas9 (K810A/K1003A/R1060A) (a.k.a. eSpCas9 1.0), and SpCas9 (K848A/K1003A/R1060A) (a.k.a. eSPCas9 1.1) are employed in the compositions. The disclosure is by no means limited to these variants, and encompasses all Cas9 variants (Slaymaker, E M. et al. (2015)).

In some embodiments, the mutant Cas9 comprises one or more mutations that alter PAM specificity (Kleinstiver et al., 2015, Nature, 523 (7561): 481-485; Kleinstiver et al., 2015, Nat Biotechnol, 33 (12): 1293-1298). In some embodiments, the mutant Cas9 comprises one or more mutations that alter the catalytic activity of Cas9, including but not limited to D10A in RuvC and H840A in HNH (Cong et al., 2013; Science 339:919-823, Gasiubas et al,, 2012;PNAS 109: E2579-2586 Jinek et al; 2012; Science 337:816-821).

The present disclosure also includes another type of enhanced specificity Cas9variant, “high fidelity” spCas9 variants (HF-Cas9) (Kleinstiver, B. P. et al., 2016, Nature. DOI: 10.1038/naturel6526).

Phage-encoded CRISPR systems: In certain embodiments, the CRISPR systems comprise phage encoded CRISPR systems. Using metagenomic analysis of microbial samples isolated from soil, aquatic, human, and animal microbiomes, the widespread occurrence of diverse, compact CRISPR-Cas systems encoded in phage genomes, was reported demonstrating an unexpected biological reservoir of anti-viral machinery within infectious agents (Basem Al-Shayeb et al., Diverse virus-encoded CRISPR-Cas systems include streamlined genome editors, Cell, Volume 185, Issue 24, 2022, Pages 4574-4586.e16, ISSN 0092-8674, doi.org/10.1016/j.cell.2022.10.020). Phage-encoded CRISPR-Cas systems include members of all six CRISPR types (types I-VI) as defined by bacterially encoded examples. New evidence was found for new or alternative modes of nucleic acid interference involving phage-encoded type I, III, IV, and VI systems. In addition, the phage and phage-like sequences result in a several-fold expansion of CRISPR-Cas9 and -Cas12 enzymes belonging to the type II and type V families that are widely deployed for genome editing applications. Cash, one of the most divergent in sequence of the phage-encoded type V enzymes identified, was found to have robust biochemical activity as an RNA-guided double-stranded DNA (dsDNA) cutter. Its cryoelectron microscopy (cryo-EM)-determined molecular structure explains its use of a natural single-guide RNA for DNA binding, and cell-based experiments demonstrated robust endogenous genome editing activity in plant and human cells. The compact architecture of Cash and other phage-encoded CRISPR-Cas proteins holds significant promise for vector-based and direct delivery into cells for wide-ranging biotechnological applications.

At least two aspects of phage-encoded CRISPR systems differ notably from cellular systems, highlighting the versatility of these pathways and also the potential for phage-mediated functional evolution. First, some RNA-targeting type III and type VI systems recognize abundant or essential transcripts of competing phages, and type III systems retain the catalytic residues required to cleave ssDNA and enable nicking of the target DNA, but lack components used for non-specific transcript cleavage following RNA target recognition. The absence of these components, which trigger abortive infection by analogous host-encoded systems, suggests that some phages prefer to avoid self-destruction of transcripts or induction of a dormant state in the host, both of which may be disadvantageous to the phage life cycle. Consistent with this idea, the minimal trans-cutting of ssDNA and RNA observed for Casλ implies limited ability to target single-stranded replication intermediates of MGEs. A second important difference between cellular and phage-encoded CRISPR systems is the absence of a processive nuclease, such as Cas3, in some phage type I systems. Together with the presence of CysH, which may be recruited as a putative effector in type IV systems, these observations suggest alternate outcomes to nucleic acid-targeting by these phage-encoded pathways. In particular, the lack of a Cas3 nuclease in type I systems targeting plasmid-like elements suggests a gene silencing mechanism that precludes DNA cutting. Such phage-based type I systems could assist the activity of the co-occurring CasΦ systems that are found in the same genome. Because the targeted plasmid-like elements harbor restriction enzymes and retron-based anti-phage defense systems that could limit the infectivity of the CRISPR-encoding phage, coordinated activities of orthogonal CRISPR systems could assist competition between mobile elements.

Phage genomes are a natural reservoir of miniature single-effector CRISPR-Cas systems, including DNA targeting type II and type V enzymes belonging to the Cas9 and Cas12 superfamilies. Greek nomenclature was used to indicate the phage origins of Casμ, CasΩ, and Casλ, extending the naming convention established by phage-encoded Cas@ (Basem Al-Shayeb et al., Diverse virus-encoded CRISPR-Cas systems include streamlined genome editors, Cell, Volume 185, Issue 24, 2022, Pages 4574-4586.e16). In contrast to the prevalence of multi-subunit type I and type III CRISPR systems in prokaryotic genomes, the notable abundance of miniature Cas12-family enzymes in phages may reflect the size restriction of many phage genomes. Because phages evolve quickly, they serve as important sources of new, divergent, or hypercompact CRISPR systems. Some of these, such as Casλ, bear sufficient sequence-level divergence to cluster separately from Cas12 and Cas9 systems and obscure a direct evolutionary relationship with known Cas superfamilies. Nonetheless, Casλ's structure, domain composition, and biochemical mechanism are similar to other type V enzymes. This finding implies that within phage genomes, distinct type V nucleases may have evolved multiple times from ancestral transposon-encoded TnpB families, which also function as RNA-guided nucleases. Despite being from different clades of phages and having divergent sequences and domain organizations, a convergent evolution of Cas12-like architecture in the Casλ and CasΦ protein structures was observed. In addition, both can process their own pre-crRNA and rely on the same RuvC active site used for DNA cleavage for this activity. This extreme compression of enzymatic activities within one active site has not been observed for bacterially encoded CRISPR-Cas proteins. Nonetheless, the phage-encoded enzymes diverge functionally from one another in other ways, including guide RNA structure and maturation process. This may reflect the interplay between rapid phage evolution, which generates diversity, and selective pressure to maintain CRISPR compatibility in a variety of host environments over time, which favors pathway conservation. In both cases, phages that encode their own Cas variants that do not rely on host factors may eliminate the possibility that the ongoing evolution of essential host proteins or cofactors will result in incompatibility with phage-encoded anti-viral systems.

Base Editing: Base editing is a CRISPR-Cas9-based genome editing technology that allows the introduction of point mutations in the DNA without generating DSBs. Two major classes of base editors have been developed: cytidine base editors or CBEs allowing C>T conversions and adenine base editors or ABEs allowing A>G conversions. In order to improve the efficiency of site-directed mutagenesis, base editing systems containing dCas9 coupled with cytosine deaminase (cytidine base editor, CBE) or adenosine deaminase (adenine base editor, ABE) have been developed (Komor A. C., et al. Programmable editing of a target base in genomic DNA without double-stranded DNA cleavage. Nature. 2016;533:420-424. Gaudelli N. M., et al. Programmable base editing of A·T to G·C in genomic DNA without DNA cleavage. Nature. 2017;551:464-471). It can introduce C·G to T·A or A·T to G·C point mutations into the editing window of the sgRNA target sites without double-stranded DNA cleavage. Since base editing systems avoid the generation of random insertions or deletions to a great extent, the results of gene mutation are more predictive. At present, base editing systems have been widely used in various cell lines, human embryos, bacteria, plants and animals for efficient site-directed mutagenesis, which may have broad application prospects in basic research, biotechnology and gene therapy. In theory, 3956 gene variants existing in Clin var database could be repaired by base substitution of C-T or G-A (Landrum M. J., et al. ClinVar: public archive of interpretations of clinically relevant variants. Nucl Acids Res. 2016;44: D862-D868).

RNA editing system: In addition to editing DNA, CRISPR-Cas systems can also edit RNA. Class 2 Type VI CRISPR-Cas13 systems contain a single RNA-guided Cas13 protein with ribonuclease activity, which can bind to target single-stranded RNA (ssRNA) and specifically cleave the target (Abudayyeh O. O., et al. RNA targeting with CRISPR-Cas13. Nature. 2017;550:280-284). To date, four Cas13 proteins have been identified: Cas13a (also known as C2c2), Cas13b, Cas13c and Cas13d (O'Connell M. R. Molecular mechanisms of RNA targeting by Cas13-containing Type VI CRISPR-Cas systems. J Mol Biol. 2019;431:66-87). They have successfully been applied in RNA knockdown, transcript labeling, splicing regulation and virus detection. Later, Feng Zhang et al. developed two RNA base editing systems (REPAIR system, enables A-to-I (G) replacement; RESCUE system, enables C-to-U replacement) by fusing catalytically inactivated Cas13 (dCas13) with the adenine/cytidine deaminase domain of ADAR2 (adenosine deaminase acting on RNA type 2) (Cox D.B.T. et al., RNA editing with CRISPR-Cas13. Science. 2017;358:1019-1027. Abudayyeh O. O. et al. A cytosine deaminase for programmable single-base RNA editing. Science. 2019;365:382-386).

Compared with DNA editing, RNA editing has the advantages of high efficiency and high specificity. Furthermore, it can make temporary, reversible genetic edits to the genome, avoiding the potential risks and ethical issues caused by permanent genome editing. At present, RNA editing has been widely used for pre-clinical studies of various diseases, which opens a new era for RNA level research, diagnosis and treatment.

Applications of CRISPR-Cas systems in gene therapy: Gene therapy refers to the introduction of foreign genes into target cells to treat specific diseases caused by mutated or defective genes (Naldini L. Gene therapy returns to centre stage. Nature. 2015;526:351-360). Target cells of gene therapy are mainly divided into two categories: somatic cells and germ line cells. Traditional gene therapy is usually carried out by homologous recombination or lentiviral delivery. Nevertheless, the efficiency of homologous recombination is low, and lentiviral vectors are randomly inserted into the recipient genome, which may bring potential security risks to clinical applications. Currently, with the rapid development of CRISPR-Cas systems, they have been widely applied in gene therapy for treating various of human diseases, monogenic diseases, infectious diseases, cancer, etc. Furthermore, some CRISPR-mediated genome-editing therapies have already reached the stage of clinical testing. Ongoing clinical trials of gene therapy using genome-editing technology, including ZFN, TALEN and CRISPR-Cas systems are summarized by Y. Xu et al. (Comput Struct Biotechnol J. 2020; 18:2401-2415).

Guide RNAs: A gRNA includes a mature crRNA that contains about 20 base pairs (bp) of unique target sequence (called spacer) and a trans-activated small RNA (tracrRNA) that serves as a guide for ribonuclease Ill-aided processing of pre-crRNA. The crRNA: tracrRNA duplex directs endonuclease to target sequence via complementary base pairing between the spacer on the crRNA and the complementary sequence (called protospacer) on the target sequence. In the present disclosure, the crRNA and tracrRNA can be expressed separately or engineered into an artificial fusion gRNA via a synthetic stem loop (AGAAAU) to mimic the natural crRNA/tracrRNA duplex. Such gRNA can be synthesized or in vitro transcribed for direct RNA transfection or expressed from U6 or H1-promoted RNA expression vector.

Further, the disclosure encompasses an isolated nucleic acid (e.g., gRNA) having substantial homology to a nucleic acid disclosed herein. In certain embodiments, the isolated nucleic acid has at least 75%, 80%, 85%, 90%, 95%, 96%, 97%, 98%, or 99% sequence homology with a nucleotide sequence of a gRNA described elsewhere herein.

In the compositions of the present disclosure, each gRNA includes a sequence that is complementary to a target sequence in bag3. The exemplary target is bag3. In certain embodiments, a gRNA sequence has at least a 75%, 80%, 85%, 90%, 95%, 96%, 97%, 98%, or 99% sequence identity to each of SEQ ID NOS: 1, 2, or 3. In certain embodiments, a gRNA sequence bas at least a 80%, 85%, 90%, 95%, 96%, 97%, 98%, or 99% sequence identity sequence identity to the complementary sequence of to each of SEQ ID NOS: 1, 2, or 3. In certain embodiments, the gRNAs comprise SEQ ID NOS: 1, 2, or 3. In certain embodiments, the gRNAs are complementary to SEQ ID NOS: 1, 2, or 3.

Guide RNA sequences according to the present disclosure can be sense or antisense sequences. The specific sequence of the gRNA may vary, but, regardless of the sequence, useful guide RNA sequences will be those that minimize off-target effects while achieving high efficiency and specificity of the target gene. The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs. When the compositions are administered in an expression vector, the guide RNAs can be encoded by a single vector. Alternatively, multiple vectors can be engineered to each include two or more different guide RNAs. Useful configurations will result in the excision of bag3 sequences between cleavage sites. The excised region can vary in size from a single nucleotide to several thousand nucleotides.

The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35,about 36, about 37, about 38, about 39, about 40, about 45, about 50, about 55, about 60 or more nucleotides. In certain embodiments the sequence of the gRNA that is substantially complementary to the target is about 10-30 nucleotides in length. In certain embodiments, the gRNA comprises a nucleotide sequence that binds to the desired target sequence in the sample. For example, in certain embodiments, the gRNA comprises a nucleotide sequence that is substantially complementary to the target sequence, and thus binds to the target sequence.

In the CRISPR-Cas system derived from S. pyogenes, the target DNA typically immediately precedes a 5′-NGG proto-spacer adjacent motif (PAM). Other Cas9 orthologs may have different PAM specificities. For example, Cas9 from S. thermophilus requires 5′-NNAGAA for CRISPR-1 and 5′-NGGNG for CRISPR-3 and Neisseria meningiditis requires 5′-NNNNGATT). The specific sequence of the guide RNA may vary, but, regardless of the sequence, useful guide RNA sequences will be those that minimize off-target effects while achieving high efficiency editing of bag3 target sequence(s). The specific sequence of the guide RNA may vary, but, regardless of the sequence, useful guide RNA sequences will be those that minimize off-target effects while achieving high efficiency editing of bag3. The length of the guide RNA sequence can vary from about 20 to about 60 or more nucleotides, for example about 20, about 21, about 22, about 23, about 24, about 25, about 26, about 27, about 28, about 29,about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome, include bioinformatic screening using target sequence+NGG target-selection criteria to exclude off-target human transcriptome or (even rarely) untranslated-genomic sites, and WGS, Sanger sequencing and SURVEYOR assay, to identify and exclude potential off-target effects. Algorithms, such as CRISPR Design Tool (CRISPR Genome Engineering Resources; Broad Institute) can be used to identify target sequences with or near requisite PAM sequences as defined by the type of Cas peptide (e.g. Cas13, Cas9, Cas9 variant, Cpfl) used. In certain embodiments, the composition comprises multiple different gRNAs, each targeted to a different target sequence. In certain embodiments, this multiplexed strategy provides for increased efficacy. In some embodiments, the compositions described herein utilize about 1 gRNA to about 6 gRNAs. In some embodiments, the compositions described herein utilize at least about 1 gRNA. In some embodiments, the compositions described herein utilize at most about 6 gRNAs. In some embodiments, the compositions described herein utilize about 1gRNA to about 2 gRNAs, about 1 gRNA to about 3 gRNAs, about 1 gRNA to about 4 gRNAs, about 1 gRNA to about 5 gRNAs, about 1 gRNA to about 6 gRNAs, about 2 gRNAs to about 3 gRNAs, about 2 gRNAs to about 4 gRNAs, about 2 gRNAs to about 5 gRNAs, about 2 gRNAs to about 6 gRNAs, about 3 gRNAs to about 4 gRNAs, about 3 gRNAs to about 5 gRNAs, about 3 gRNAs to about 6 gRNAs, about 4 gRNAs to about 5 gRNAs, about 4 gRNAs to about 6 gRNAs, or about 5 gRNAs to about 6 gRNAs. In some embodiments, the compositions described herein utilize about 1 gRNA, about 2 gRNAs, about 3 gRNAs, about 4 gRNAs, about 5 gRNAs, or about 6 gRNAs.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the guide RNA sequences or in a separate vector. In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington DC). Modified RNA components include the following: 2′-O-methylcytidine; N⁴-methylcytidine; N⁴-2′-O-dimethylcytidine; N⁴-acetylcytidine; 5-methylcytidine; 5, 2′-O-dimethyl cytidine; 5-hydroxymethyl cytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl) uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-0-methyluridine; 5-(carboxyhydroxymethyl) uridine; 5-(carboxyhydroxymethyl) uridinemethyl ester; 5-aminomethyl-2-thiouridine; 5-methylaminomethyluridine; 5-methylaminomethy 1-2-thiouridine; 5-methylaminomethy 1-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis—hydroxyisopentenyl)-adenosine; N⁶-glycinylcarbamoyl) adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′0-methyl inosine; 1-methyl inosine; 1; 2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N²;7-dimethyl guanosine; N²; N²; 7-trimethyl guanosine; wyosine; methylwyosine; undermodified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachacosine [also called 7-formamido-7-deazaguanosine]; and 7-aminomethyl-7-deazaguanosine.

Isolated nucleic acid molecules can be produced by standard techniques. For example, PCR techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the disclosure also can be obtained by mutagenesis of, e.g., a naturally occurring portion of a Cas13-encoding DNA.

In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalently couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments all of the inter-nucleoside linkers of the gRNA, or contiguous nucleotide sequence thereof, are modified. For example, in some embodiments the inter-nucleoside linkage comprises Sulphur(S), such as a phosphorothioate inter-nucleoside linkage.

Modifications to the ribose sugar or nucleobase can also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than Hor-OH at the 2′ position (2′ substituted nucleoside) or comprises a 2′ linked biradicle and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region (e.g, SEQ ID NOS: 1, 2, or 3) and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence. gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the crRNA or traerRNA comprises a modified sequence. In certain embodiments, the crRNA or tracrRNA comprises at least 1, 2, 3, 4, 5, 10, or 15 modified bases (e.g. a modified native base sequence).

Complementary, as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double stranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80%, 85%, 90%, or 95% sequence identity. In certain embodiments, sequence identity includes in addition to non-identical nucleobases, sequences comprising insertions and/or deletions.

The nucleic acid of the disclosure, including the RNA (e.g., crRNA, tracrRNA, gRNA) or nucleic acids encoding the RNA, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid e.g., using automated DNA synthesis in the 3′ to 5′ direction using phosphoramidite technology) or as a series of oligonucleotides. Isolated nucleic acids of the disclosure also can be obtained by mutagenesis of, e.g., a naturally occurring portion erRNA, tracrRNA, RNA-encoding DNA, or of a Cas-encoding DNA.

In certain embodiments, the isolated RNA are synthesized from an expression vector encoding the RNA molecule, as described in detail elsewhere herein.

### Nucleic Acids and Vectors

In some embodiments, the composition of the disclosure comprises an isolated nucleic acid encoding one or more elements of the CRISPR-Cas system described herein. For example, in some embodiments, the composition comprises an isolated nucleic acid encoding at least one guide nucleic acid (e.g., gRNA). In some embodiments, the composition comprises an isolated nucleic acid encoding a Cas peptide, or functional fragment or derivative thereof. In some embodiments, the composition comprises an isolated nucleic acid encoding at least one guide nucleic acid (e.g., gRNA) and encoding a Cas peptide, or functional fragment or derivative thereof. In some embodiments, the composition comprises an isolated nucleic acid encoding at least one guide nucleic acid (e.g., gRNA) and further comprises an isolated nucleic acid encoding a Cas peptide, or functional fragment or derivative thereof.

In some embodiments, the composition comprises at least one isolated nucleic acid encoding a gRNA, where the gRNA is substantially complementary to a target sequence of the bag3, as described elsewhere herein. In some embodiments, the composition comprises at least one isolated nucleic acid encoding a gRNA, where the gRNA is complementary to a target sequence having at least 75%, 80%, 85%, 90%, 95%, 96%, 97%, 98%, or 99% sequence homology to a target sequence described herein.

In some embodiments, the composition comprises at least one isolated nucleic acid encoding a Cas peptide described elsewhere herein, or a functional fragment or derivative thereof. In some embodiments, the composition comprises at least one isolated nucleic acid encoding a Cas peptide having at least 75%, 80%, 85%, 90%, 95%, 96%, 97%, 98%, or 99% amino acid sequence homology with a Cas peptide described elsewhere herein.

The isolated nucleic acid may comprise any type of nucleic acid, including, but not limited to DNA and RNA. For example, in some embodiments, the composition comprises an isolated DNA, including for example, an isolated cDNA, encoding a gRNA or peptide of the disclosure, or functional fragment thereof. In some embodiments, the composition comprises an isolated RNA encoding a peptide of the disclosure, or a functional fragment thereof. The isolated nucleic acids may be synthesized using any method known in the art.

The present disclosure can comprise use of a vector in which the isolated nucleic acid described herein is inserted. The art is replete with suitable vectors that are useful in the present disclosure. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al. BioTechniques, 34:167-171 (2003). A large variety of such vectors is known in the art and is generally available.

In brief summary, the expression of natural or synthetic nucleic acids encoding an RNA and/or peptide is typically achieved by operably linking a nucleic acid encoding the RNA and/or peptide or portions thereof to a promoter and incorporating the construct into an expression vector. The vectors to be used are suitable for replication and, optionally, integration in eukaryotic cells. Typical vectors contain transcription and translation terminators, initiation sequences, enhancers and promoters useful for regulation of the expression of the desired nucleic acid sequence. The vectors of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

The isolated nucleic acid of the disclosure can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

Further provided are nucleic acids encoding the CRISPR-Cas systems described herein. Provided herein are adeno-associated virus (AAV) vectors comprising nucleic acids encoding the CRISPR-Cas systems described herein. In certain instances, an AAV vector includes to any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. In certain instances, an AAV vector includes an AAV type viral particle (or virion) comprising a nucleic acid encoding a protein of interest (e.g. CRISPR-Cas systems described herein). In some embodiments, as further described herein, the AAVs disclosed herein are be derived from various serotypes, including combinations of serotypes (e.g., “pseudotyped” AAV) or from various genomes (e.g., single-stranded or self-complementary). In some embodiments, the AAV vector is a human serotype AAV vector. In such embodiments, a human serotype AAV is derived from any known serotype, e.g., from AAV1, AAV2, AAV4, AAV6, or AAV9. In some embodiments, the serotype is AAV1, AAV2, AAV3, AAV4, AAV5, AAV6, AAV7, AAV8, AAV9, AAV10, AAV11, AAVDJ, or AAVDJ/8.

In some embodiments, the composition includes a vector derived from an adeno-associated virus (AAV). AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

A variety of different AAV capsids have been described and can be used, although AAV which preferentially target the liver and/or deliver genes with high efficiency are particularly desired. The sequences of the AAV8 are available from a variety of databases. While the examples utilize AAV vectors having the same capsid, the capsid of the gene editing vector and the AAV targeting vector are the same AAV capsid. Another suitable AAV is, e.g., rh10 (WO 2003/042397). Still other AAV sources include, e.g., AAV9 (see, for example, U.S. Pat. No. 7,906,111; US 2011-0236353-A1), and/or hu37 (see, e.g., U.S. Pat. No. 7,906, 111; US 2011-0236353-A1), AAV1, AAV2, AAV3, AAV4, AAV5, AAV6, AAV6.2, AAV7, AAV8, (U.S. Pat. No. 7,790,449; U.S. Pat. No. 7,282,199, WO 2003/042397; WO 2005/033321, WO 2006/110689; U.S. Pat. No. 7,790,449; U.S. Pat. No. 7,282,199; U.S. Pat. No. 7,588,772). Still other AAV can be selected, optionally taking into consideration tissue preferences of the selected AAV capsid.

In some embodiments, AAV vectors disclosed herein include a nucleic acid encoding a CRISPR-Cas systems described herein. In some embodiments, the nucleic acid also includes one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid comprises a promoter region operably linked to the coding sequence to cause or improve expression of the protein of interest in infected cells. Such a promoter can be ubiquitous, cell-or tissue-specific, strong, weak, regulated, chimeric, etc., for example, to allow efficient and stable production of the protein in the infected tissue. In certain embodiments, the promoter is homologous to the encoded protein, or heterologous, although generally promoters of use in the disclosed methods are functional in human cells. Examples of regulated promoters include, without limitation, Tet on/off element containing promoters, rapamycin-inducible promoters, tamoxifen-inducible promoters, and metallothionein promoters. In certain embodiments, other promoters used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the phosphoglycerate kinase (PGK) promoter and the b-actin promoter.

In some embodiments, the recombinant AAV vector comprises packaged within an AAV capsid, a nucleic acid, generally containing a 5′ AAV ITR, the expression cassettes described herein and a 3′ AAV ITR. As described herein, in some embodiments, an expression cassette contains regulatory elements for an open reading frame(s) within each expression cassette and the nucleic acid optionally contains additional regulatory elements. The AAV vector, in some embodiments, comprises a full-length AAV 5′ inverted terminal repeat (ITR) and a full-length 3′ ITR. A shortened version of the S′ ITR, termed AITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (see, for example, D M McCarty et al., “Self-complementary recombinant adeno-associated virus (scAAV) vectors promote efficient transduction independently of DNA synthesis”, Gene Therapy, (August 2001); see also, for example, U.S. Pat. Nos. 6,596,535; 7,125,717; and 7,456,683). Where a pseudotyped AAV is to be produced, the ITRs are selected from a source which differs from the AAV source of the capsid. For example, in some embodiments, AAV2 ITRs are selected for use with an AAV capsid having a particular efficiency for a selected cellular receptor, target tissue or viral target. In some embodiments, the ITR sequences from AAV2, or the deleted version thereof (AITR), are used for convenience and to accelerate regulatory approval (i.e. pseudotyped). In some embodiments, a single-stranded AAV viral vector is used.

Methods for generating and isolating AAV viral vectors suitable for delivery to a subject are known in the art (see, for example, U.S. Pat. No. 7,790,449; U.S. Pat. No. 7,282,199; WO 2003/042397; WO 2005/033321, WO 2006/110689; and U.S. Pat. No. 7,588,772 B2, U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV-the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors.

The CRISPR-Cas systems, and/or any of the present RNAs, for instance a guide RNA, can be delivered using adeno associated virus (AAV), lentivirus, adenovirus or other viral vector types, or combinations thereof. An endonuclease and one or more guide RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery can be either via a single dose, or multiple doses. One skilled in the art understands that the actual dosage to be delivered herein can vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some embodiments. The adenovirus vector results in a shorter term expression (e.g., less than about a month) than adeno-associated virus, in some embodiments, may exhibit much longer expression. The particular vector chosen will depend upon the target cell and the condition being treated.

In certain embodiments, the vector also includes conventional control elements which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. One example of a suitable promoter is the immediate early cytomegalovirus (CMV) promoter sequence. This promoter sequence is a strong constitutive promoter sequence capable of driving high levels of expression of any polynucleotide sequence operatively linked thereto. The Rous sarcoma virus (RSV) and MMT promoters may also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUCI 18, pBR322, or other known plasmid vectors, that includes, for example, an E. coli origin of replication.

Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatinine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In some embodiments, the vector of the present disclosure comprises one or more enhancers to boost transcription of the gene present within the vector.

In order to assess the expression of the nucleic acid and/or peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, micro injection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

In certain embodiments, the composition comprises a cell genetically modified to express one or more isolated nucleic acids and/or peptides described herein. For example, the cell may be transfected or transformed with one or more vectors comprising an isolated nucleic acid sequence encoding a gRNA and/or a Cas peptide. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. IPS cells, in particular, can be established from self-derived somatic cells, and therefore are not likely to cause ethical and social issues, in comparison with ES cells which are produced by destruction of embryos. Further, iPS cells, which are a selfderived cell, make it possible to avoid rejection reactions, which are the biggest obstacle to regenerative medicine or transplantation therapy.

### Delivery Vehicles

Delivery vehicles as used herein, include any types of molecules for delivery of the compositions embodied herein, both for in vitro or in vivo delivery. Examples, include, without limitation: expression vectors, nanoparticles, colloidal compositions, lipids, liposomes, nanosomes, carbohydrates, organic or inorganic compositions and the like. Any suitable method can be used to deliver the compositions to the subject. In certain embodiments, the nucleases, e.g. CRISPR/Cas, or the genes encoding the nuclease may be delivered to systematic circulation or may be delivered or otherwise localized to a specific tissue type. The nuclease or gene encoding the nuclease may be modified or programmed to be active under only certain conditions such as by using a tissue-specific promoter so that the encoded nuclease is preferentially or only transcribed in certain tissue types.

In some embodiments, a delivery vehicle is an expression vector, wherein the expression vector encodes a desired nucleic acid sequence. In certain embodiments, the vector comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in subject's genome. The endonuclease and the guide RNA may be co-expressed in a host cell infected by a virus.

In some embodiments, the compositions of the disclosure can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor.

In some embodiments of the disclosure, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP). Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphipathic and polar lipid component and at least one nonpolar lipid component.

Liposomes and polymerosomes can contain a plurality of solutions and compounds. In certain embodiments, the complexes of the disclosure are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21 (20): 9183-6, incorporated by reference.

In some embodiments of the disclosure, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles.

### Pharmaceutical Compositions

Certain aspects of the instant disclosure pertain to pharmaceutical compositions of the compounds of the disclosure. The pharmaceutical compositions of the disclosure typically comprise a compound of the instant disclosure and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration.

Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the instant disclosure is contemplated. Supplementary active compounds can also be incorporated into the compositions.

Therapeutic compositions typically must be sterile and stable under the conditions of manufacture and storage. The composition can be formulated as a solution, micro emulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer, or in a fat pad described herein. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, certain methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes. Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (e.g., CRISPR/Cas) preferably is formulated in the composition in a therapeutically effective amount. A “therapeutically effective amount” refers to an amount effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the instant disclosure is dictated by and directly dependent on (a) the unique characteristics of the active compound and the particular therapeutic effect to be achieved, and (b) the limitations inherent in the art of compounding such an active compound for the treatment of sensitivity in individuals.

The compound(s) of the instant disclosure can be administered in a manner that prolongs the duration of the bioavailability of the compound(s), increases the duration of action of the compound(s) and the release time frame of the compound by an amount selected from the group consisting of at least 3 hours, at least 6 hours, at least 12 hours, at least 24 hours, at least 48 hours, at least 72 hours, at least 4 days, at least 5 days, at least 6 days, at least 7 days, at least 2 weeks, at least 3 weeks, and at least a month, over that of the compound(s) in the absence of the duration-extending administration. Optionally, the duration of any or all of the preceding effects is extended by at least 30 minutes, at least an hour, at least 2 hours, at least 3 hours, at least 6 hours, at least 12 hours, at least 24 hours, at least 48 hours, at least 72 hours, at least 4days, at least 5 days, at least 6 days, at least 7 days, at least 2 weeks, at least 3 weeks or at least a month.

A compound of the instant disclosure can be formulated into a pharmaceutical composition wherein the compound is the only active agent therein. Alternatively, the pharmaceutical composition can contain additional active agents. For example, two or more compounds of the instant disclosure may be used in combination.

### Methods of Treatment

The present disclosure provides a method of treating or preventing myopathies, cancer, neurological diseases or disorders. In some embodiments, the method comprises administering to a subject in need thereof, an effective amount of a composition comprising at least one of a guide nucleic acid and a Cas peptide, or functional fragment or derivative thereof.

In some embodiments, the method comprises administering a composition comprising an isolated nucleic acid encoding at least one of: the guide nucleic acid and a Cas peptide, or functional fragment or derivative thereof. In certain embodiments, the method comprises administering a composition described herein to a subject diagnosed with a myopathies, cancer, neurological diseases or disorders, at risk for developing a myopathies, cancer, neurological diseases or disorders and the like.

Provided herein, in certain embodiments, are methods of modifying and/or editing a bag3 sequence in the genome of a cell (e.g. host cell) using the CRISPR-Cas systems or compositions described herein. Generally, of modifying and/or editing the bag3 sequence in the genome of a cell (e.g. host cell) comprises contacting a cell, or providing to the cell, a CRISPR-Cas system or composition targeting one or more regions in the bag3 gene. In some embodiments, the methods comprise removing or excising a sequence from a genome of the cell.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

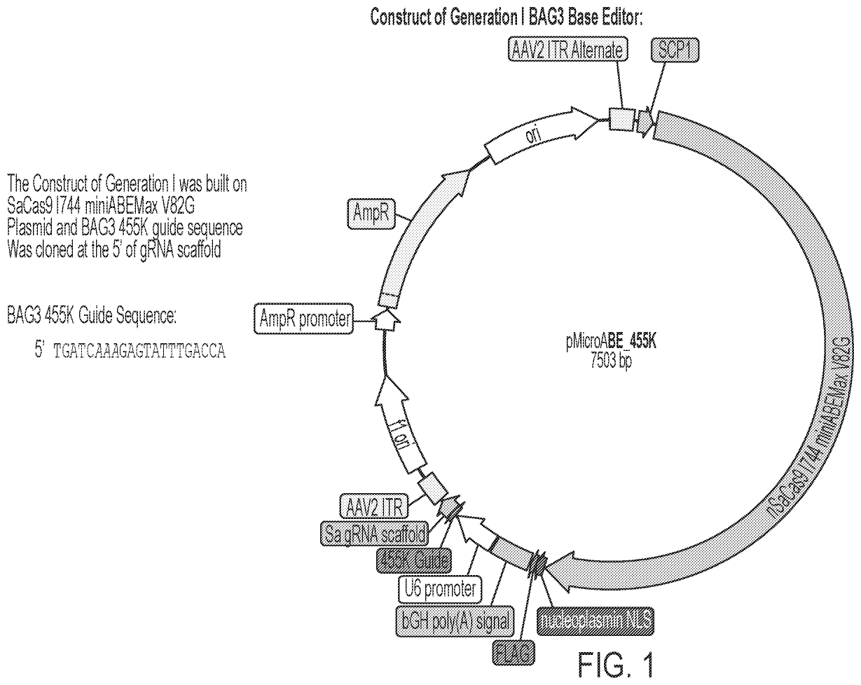

Construct of Generation I BAG3 Base Editor: The construct of generation I was built on SaCas9 1744 miniABEMax V82G Plasmid and the BAG3 455K guide sequence was cloned at the 5′ of gRNA scaffold. BAG3 455K Guide Sequence: 5′ TGATCAAAGAGTATTTGACCA. This is the first generation of base editor. The base editing elements in this construct is small enough to be packaged into AAV. FIG. 1 is a schematic representation of a construct of generation I BAG3 base editor.

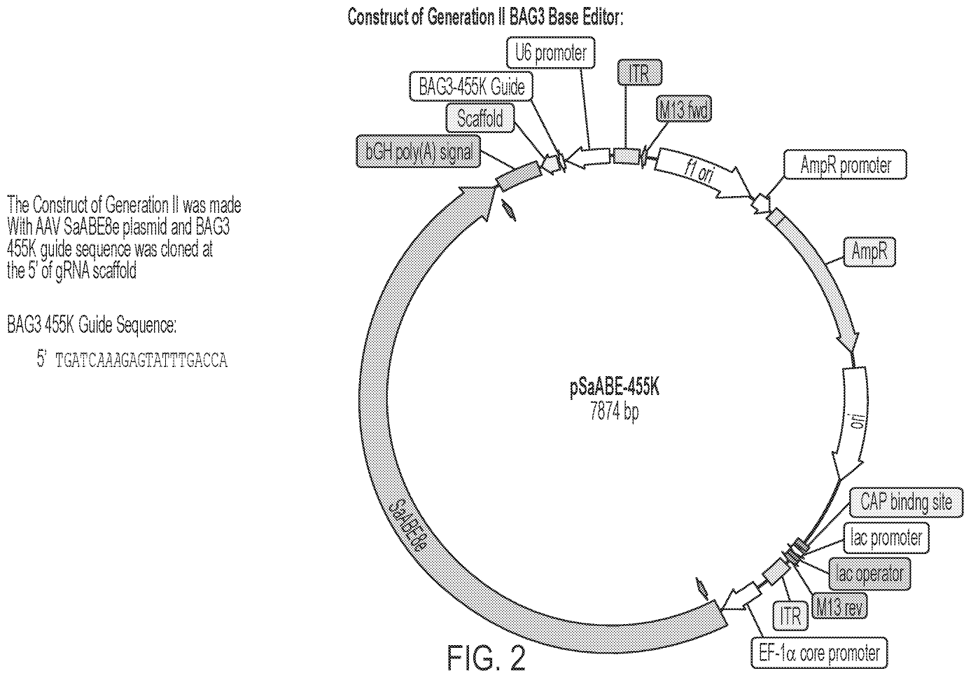

Construct of Generation II BAG3 Base Editor: The construct of generation II was made with AAV SaABESe plasmid and BAG3 455K guide sequence was cloned at the 5′ of gRNA scaffold. BAG3 455K Guide Sequence: 5′ TGATCAAAGAGTATTTGACCA. This is the 2nd generation of base editor. All the elements for base editing in this construct can be packaged into AAV. The editing efficiency is higher than the first generation BAG3 base editor shown in FIG. 1. FIG. 2 is a schematic representation of a construct of generation I BAG3 base editor.

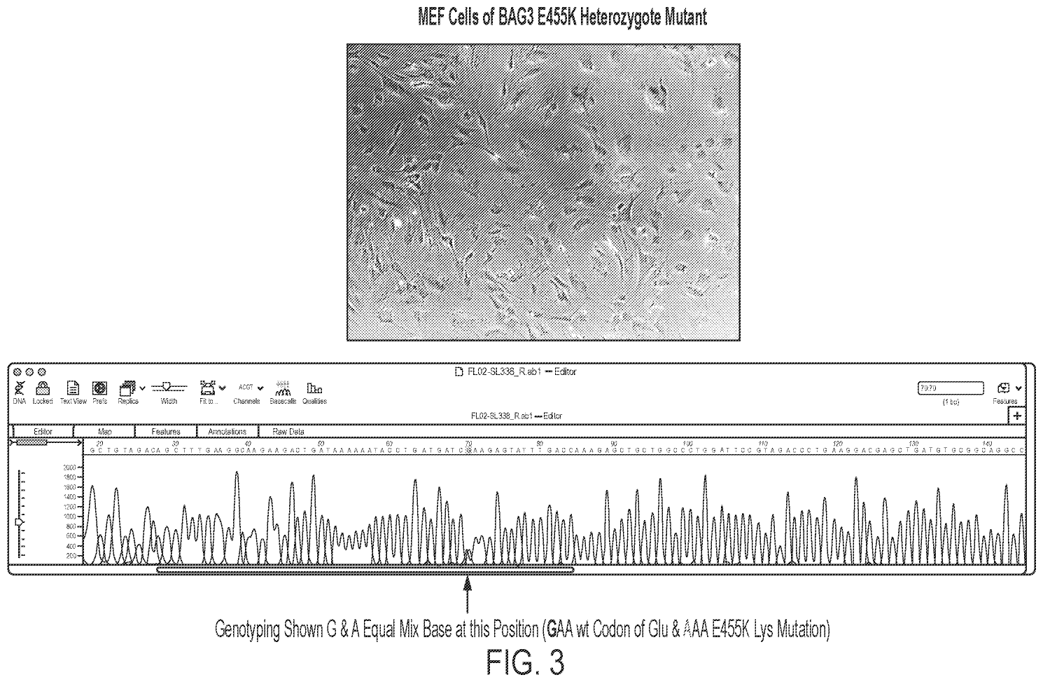

FIG. 3 shows the BAG3 E455K Heterozygote Mutant MEF cells (top panel) and the genotyping of these mutant cells (bottom panel) confirmed that half of DNA has a “A” at the position in the sequence, which is translated into a mutant amino acid, Lys, while the other half has a “G” at the same position in the sequence, which is translated into the wild type amino acid, Glu.

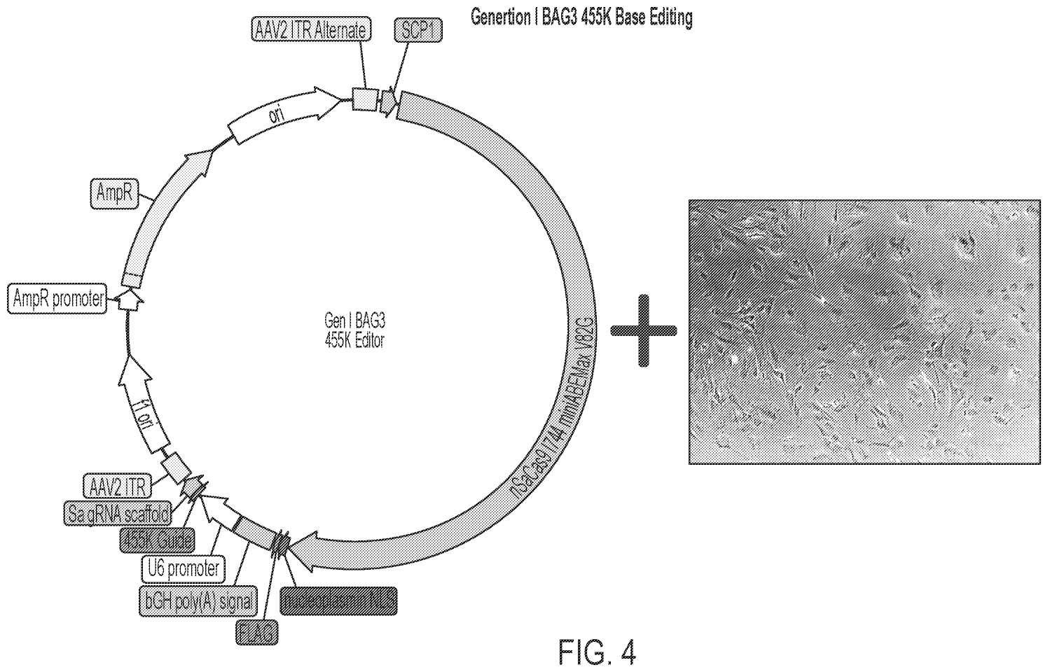

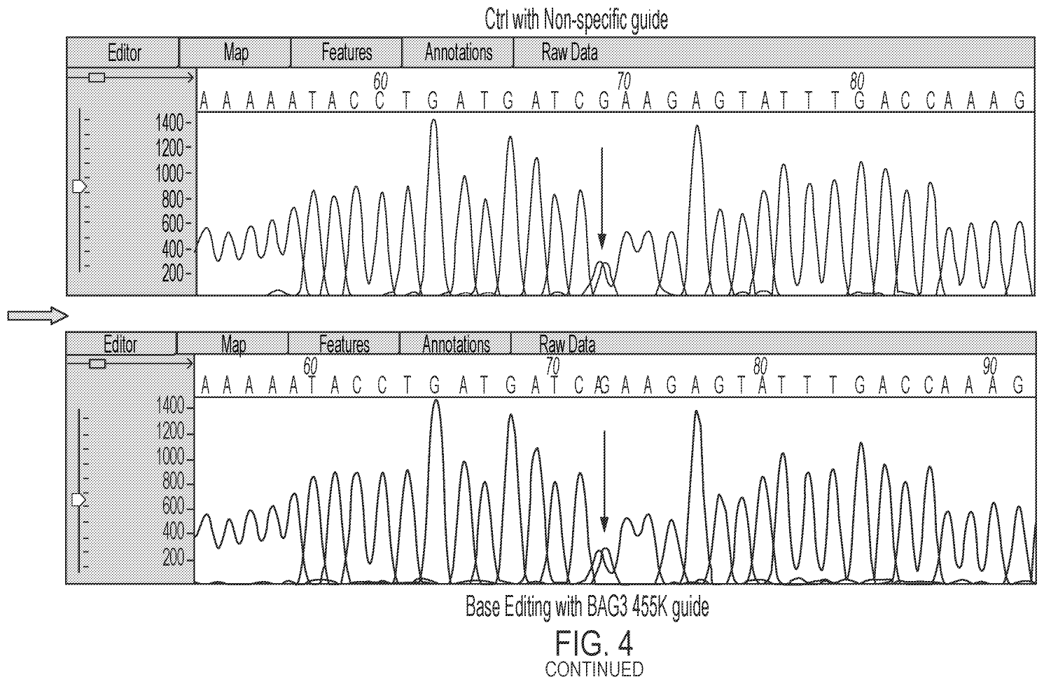

FIG. 4 shows results from BAG3 455K base editing with Generation I editor. Right panel shows the reaction of MEF Heterozygote mutant cells are edited with generation I editor, Left panel shows the genotyping of edited cells. Left/Top is the control reaction the genotyping of which shows no base change indicated by the red arrow, Left/Bottom is the reaction with Generation I 455K editor, the genotyping of which shows a little increase in G signal (estimated about 1%) indicated by the red arrow.

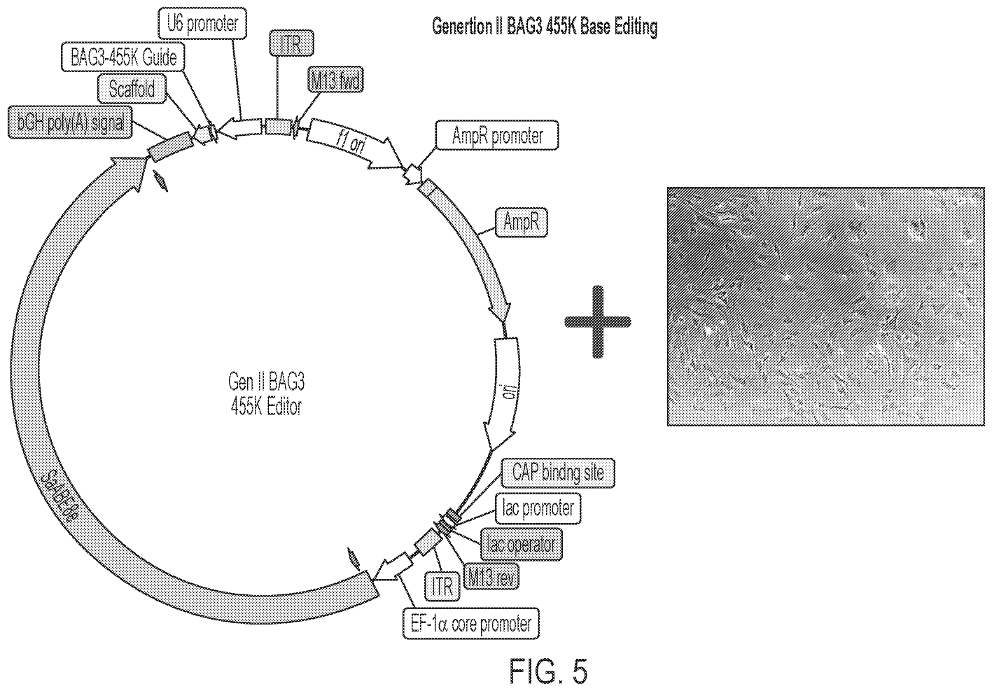

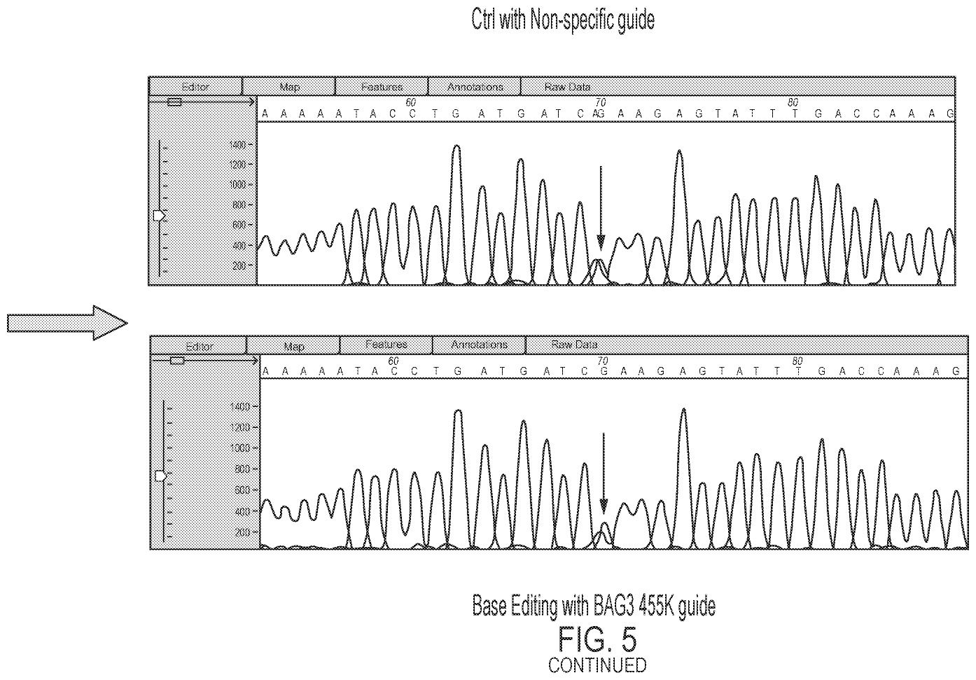

FIG. 5 shows results from BAG3 455K base editing with Generation II editor. Right panel shows the reaction of MEF Heterozygote mutant cells are edited with generation II editor, Left panel shows the genotyping of edited cells. Left/Top is the control reaction the genotyping of which shows no base change (Both “A” and “G” signals are the same as unedited cells indicated by the red arrow, Left/Bottom is the reaction with Generation II 455K editor, the genotyping of which shows a significant increase in “G” signal (Estimated about 9%) (Suggesting some of DNA was changed/edited from “A” base, the mutant to “G” base, the wild-type indicated by the red arrow.

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed:

1. A method of diagnosing and treating a subject suspected of having a myopathy comprising:
identifying in a subject's biological sample, at least one Bcl2-associated anthanogene 3 (BAG3) genetic mutation as compared to a control BAG3 nucleic acid sequence, wherein detection of certain genetic mutations is diagnostic of a myopathy,
administering to the subject a therapeutically effective amount of a gene-editing complex, wherein the gene-editing complex corrects the bag3 mutation to a wild-type bag3, thereby, treating the subject.

2. The method of claim 1, wherein the mutation comprises insertions, deletions, truncation, substitutions or combinations thereof.

3. The method of claim 2, wherein the mutations encode for a mutated BAG3 polypeptide.

4. The method of claim 3, wherein the mutation comprises an E455K.

5. The method of claim 1, wherein the gene editing complex comprises comprising at least one isolated nucleic acid sequences wherein the isolated nucleic acid sequences encode a Clustered Regularly Interspaced Short Palindromic Repeat (CRISPR)-associated endonuclease and at least one guide RNA (gRNA), the gRNA being complementary to a target sequence in the bag3 gene.

6. The method of claim 5, wherein the at least one guide RNA comprises a sequence having at least a 90% sequence identity to SEQ ID NOS: 1, 2, or 3.

7. The method of claim 5, wherein the at least one guide RNA comprises sequences SEQ ID NOS: 1, 2, or 3.

8. A method of treating cancer comprising: administering to the subject a therapeutically effective amount of an agent, wherein the agent modulates expression or amount of BAG3 molecules, proteins or peptides thereof in a target cell or tissue, as compared to a normal control, thereby treating cancer.

9. The method of claim 8, wherein increased amounts of BAG3 nucleic acids and/or BAG3 peptides as compared to normal controls are diagnostic of cancer.

10. The method of claim 9, wherein the cancer comprises melanomas, glioblastomas or adenocarcinomas.

11. The method of claim 8, wherein the agent comprises miRNA, dsDNA, IncRNA, siRNA, short hairpin RNAs (shRNAs), antisense oligonucleotide, a phosphorodiamidate morpholino oligomer (PMO), a peptide-conjugated phosphorodiamidate morpholino oligomer (PPMO), ribozymes, gene-editing complexes, or combinations thereof.

12. The method of claim 11, wherein the gene editing complex comprises comprising at least one isolated nucleic acid sequences wherein the isolated nucleic acid sequences encode a Clustered Regularly Interspaced Short Palindromic Repeat (CRISPR)-associated endonuclease and at least one guide RNA (gRNA), the gRNA being complementary to a target sequence in the bag3 gene.

13. The method of claim 12, wherein the gene-editing agent is targeted to the bag3 gene, bag3 gene regulatory elements or the combination thereof.

14. The method of claim 13, wherein the gene regulatory elements comprise promoters, enhancers, initiation codons, stop codons, polyadenylation signals or combinations thereof.

15. A method of treating preventing or treating neurodegeneration in a subject, comprising administering Bcl2-associated athanogene 3 (BAG3) polynucleotide, polypeptide and/or agents which induce BAG3 expression or function.

16. The method of claim 15, wherein the agent comprises miRNA, dsDNA, IncRNA, siRNA, short hairpin RNAs (shRNAs), antisense oligonucleotide, a phosphorodiamidate morpholino oligomer (PMO), a peptide-conjugated phosphorodiamidate morpholino oligomer (PPMO), ribozymes, gene-editing complexes, proteins or peptides thereof, peptidomimetics, small molecules, organic or inorganic compounds, synthetic or natural compounds, or combinations thereof.

17. The method of claim 16, wherein the gene editing complex comprises comprising at least one isolated nucleic acid sequences wherein the isolated nucleic acid sequences encode a Clustered Regularly Interspaced Short Palindromic Repeat (CRISPR)-associated endonuclease and at least one guide RNA (gRNA), the gRNA being complementary to a target sequence in the bag3 gene.

18. The method of claim 17, wherein the gene-editing agent is targeted to bag3 gene, bag3 gene regulatory elements or the combination thereof.

19. The method of claim 18, wherein the gene regulatory elements comprise promoters, enhancers, initiation codons, stop codons, polyadenylation signals or combinations thereof.

20. The method of claim 16, wherein the agent comprises an expression vector expressing a BAG3 protein or active fragments thereof, oligonucleotides or combinations thereof.

21. The method of claim 20, wherein the expression vector comprises a viral vector, tropic vector, a neurotropic vector, a cardiotropic vector, plasmid, or a yeast vector.

22. The method of claim 21, wherein a cardiotropic vector comprises an adenovirus vector, an adeno-associated virus vector (AAV), a coxsackie virus vector, cytomegalovirus vector, Epstein-Barr virus vector, parvovirus vector, or hepatitis virus vectors.
